(19) United States
(12) Patent Application Publication
(10) Pub. No.: US 2022/0241470 A1
(43) Pub. Date: Aug. 4, 2022

(54) FLOW PATH DEVICE AND BIOLOGICAL COMPONENT BAG SYSTEM

(71) Applicant: TERUMO KABUSHIKI KAISHA, Tokyo (JP)

(72) Inventor: Masatsugu IGARASHI, Shizuoka (JP)

(21) Appl. No.: 17/629,280

(22) PCT Filed: Jul. 17, 2020
(86) PCT No.: PCT/JP2020/027770

§ 371 Date: Jan. 21, 2022

(30) Foreign Application Priority Data
Jul. 22, 2019 (JP) 2019-134553

(51) Int. Cl. A61M 1/02

(52) U.S. Cl. CPC A61M 1/0209; A61M 2202/0439; A61M 2202/0468; A61M 1/0218

(57) ABSTRACT

A flow path device of a biological component bag system is equipped with a flow path formation member. The flow path formation member includes a first sheet and a second sheet, and flow paths are formed between the first sheet and the second sheet. The flow paths of the flow path formation member include first flow paths and a second flow path. Flow path sealed portions that join the first sheet and the second sheet to each other in a liquid-tight manner are provided on both sides of each of the flow paths within the flow path formation member.

### TECHNICAL FIELD

The present invention relates to a flow path device and a biological component bag system.

### BACKGROUND ART

In FIG. 9 of International Publication No. WO 2018/062211, a flow path device is disclosed in which a buffy coat accommodated in a plurality of bags is transferred and collected in a single pooling bag. Such a flow path device includes a plurality of branch connectors constituted by a hard material, and a plurality of tubes connected respectively to a plurality of port members of the branch connectors.

### SUMMARY OF INVENTION

In the above-described flow path device, the plurality of branch connectors and the plurality of tubes are required, and therefore, the number of component parts is relatively large. Further, since a large number of component parts (branch connectors and tubes) must be joined together, the number of joining steps increases, and there is a concern that the cost of the flow path device may increase.

The present invention has been devised taking into consideration the aforementioned problems, and has the object of providing a flow path device and a biological component bag system which are capable of achieving a reduction in cost.

A first aspect of the present invention is characterized by a flow path device including a flow path formation member in which there are formed flow paths configured to allow at least one of a biological fluid and a medicinal solution to flow therethrough, the flow path formation member including a first sheet formed by a soft material, and a second sheet formed by a soft material and that is superimposed on the first sheet, wherein the flow paths are formed between the first sheet and the second sheet, the flow paths including a plurality of first flow paths, and a single second flow path configured to communicate with the plurality of first flow paths, wherein flow path sealed portions configured to seal the first sheet and the second sheet to each other in a liquid-tight manner are provided on both sides of each of the flow paths within the flow path formation member.

A second aspect of the present invention is characterized by a biological component bag system configured to collect a desired biological component from a biological fluid, the biological component bag system including a plurality of first bags in which the biological fluid is accommodated, a flow path device to which the plurality of first bags are connected, and a second bag configured to accommodate the biological component guided from the plurality of first bags via the flow path device, wherein the flow path device includes a flow path formation member in which there are formed flow paths configured to allow at least one of the biological fluid and a medicinal solution to flow therethrough, the flow path formation member including a first sheet formed by a soft material, and a second sheet formed by a soft material and that is superimposed on the first sheet, wherein the flow paths are formed between the first sheet and the second sheet, the flow paths including a plurality of first flow paths, and a single second flow path configured to communicate with the plurality of first flow paths, wherein flow path sealed portions configured to join the first sheet and the second sheet to each other in a liquid-tight manner are provided on both sides of each of the flow paths within the flow path formation member.

According to the present invention, the flow paths (the plurality of first flow paths and the second flow path) are formed between the first sheet and the second sheet. Therefore, compared to a conventional product in which a plurality of branch connectors and a plurality of tubes are used, the number of component parts can be reduced. In addition, the flow path sealed portions that join the first sheet and the second sheet to each other in a liquid-tight manner are disposed on both sides of the liquid flow paths. Consequently, since there is no need to join a large number of component parts, the number of joining steps can be reduced. Thus, a reduction in costs can be achieved.

### BRIEF DESCRIPTION OF DRAWINGS

FIG. 1 is a schematic configuration diagram of a biological component bag system equipped with a flow path device according to an embodiment of the present invention;

FIG. 2 is a partially enlarged plan view of the flow path device shown in FIG. 1;

FIG. 3 is a cross-sectional view taken along line III-III of FIG. 2;

FIG. 4 is a flowchart for explaining a method of manufacturing the flow path device shown in FIG. 2;

FIG. 5 is an explanatory diagram of an arrangement step shown in FIG. 4;

FIG. 6A is an explanatory diagram of a joining step shown in FIG. 4, and FIG. 6B is an explanatory diagram of a blow molding step shown in FIG. 4;

FIG. 7 is a flowchart for describing a blood platelet collection method in which the biological component bag system shown in FIG. 1 is used;

FIG. 8 is a first explanatory diagram for describing the blood platelet collection method in which the biological component bag system shown in FIG. 1 is used;

FIG. 9 is a second explanatory diagram for describing the blood platelet collection method in which the biological component bag system shown in FIG. 1 is used;

FIG. 10 is a partially enlarged plan view of a flow path device according to a first modification;

FIG. 11 is a flowchart for explaining a method of manufacturing the flow path device shown in FIG. 10;

FIG. 12 is a partially enlarged plan view of a flow path device according to a second modification;

FIG. 13 is a flowchart for explaining a method of manufacturing the flow path device shown in FIG. 11; and

FIG. 14 is a partially enlarged plan view of a flow path device according to a third modification.

### DESCRIPTION OF EMBODIMENTS

Hereinafter, a preferred embodiment of a flow path device and a biological component bag system according to the present invention will be presented and described in detail below with reference to the accompanying drawings.

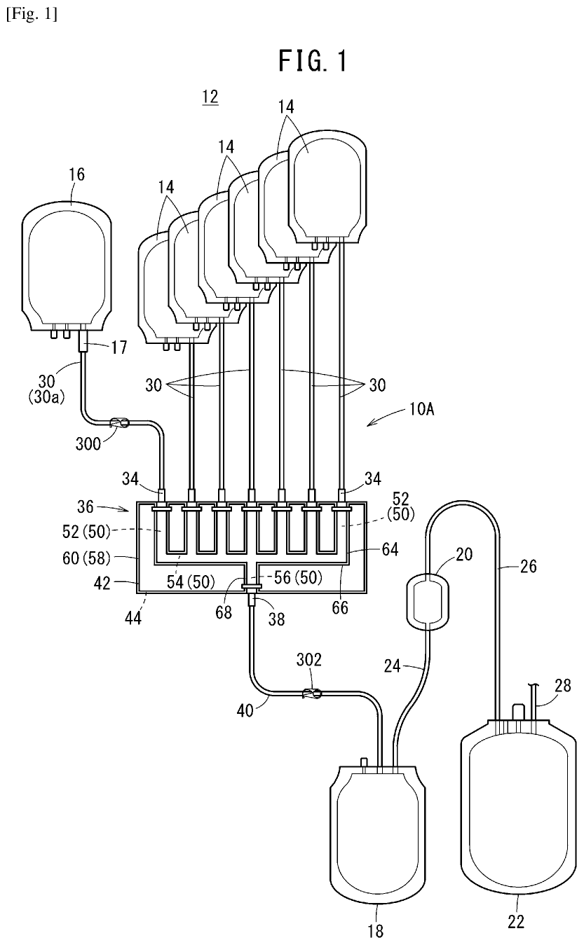

As shown in FIG. 1, the biological component bag system 12 according to one embodiment of the present invention is a system that transfers and collects a buffy coat accommodated in a plurality of BC bags 14 into a pooling bag 18, and thereafter, removes leukocytes from the buffy coat in the pooling bag 18 in order to obtain a blood platelet (blood platelet preparation). However, the biological component bag system 12 is not limited to such an example in which blood platelets are collected from a buffy coat, and may be any system that serves to collect a desired biological component from a biological fluid.

The biological component bag system 12 includes the plurality of BC bags 14 (first bags), a medicinal solution bag 16, a flow path device 10A, the pooling bag 18 (second bag), a filter 20, and a blood platelet bag 22 (third bag).

The BC bags 14 are constituted in bag shapes, for example, by overlapping sheet materials possessing flexibility and made of a soft resin such as polyvinyl chloride, polyolefin, or the like, and fusion bonding (heat fusion bonding, high frequency fusion bonding) or adhering the sheet materials to each other at sealed portions on peripheral edges thereof. Moreover, the medicinal solution bag 16, the pooling bag 18, and the blood platelet bag 22 are configured in the same bag shape as the BC bags 14.

The BC bags 14 contain the buffy coat. The buffy coat inside the BC bags 14 is obtained, for example, by centrifuging blood (whole blood) containing a plurality of blood components into blood plasma, the buffy coat, and concentrated red blood cells. However, the method of collecting the buffy coat is not limited to such a method, and may be changed as appropriate. Moreover, a small amount of the blood plasma and the concentrated red blood cells remain in the buffy coat inside the BC bags 14. In the present embodiment, six BC bags 14 are provided.

A blood platelet preservation solution (PAS: Platelet Additive Solution) which serves as the medicinal solution (medicinal fluid) is accommodated in the medicinal solution bag 16. As examples of the platelet additive solution, there may be cited Composol, Tsol, Intersol, Plasma LiteA, SSP, SSP+, Seto solution, M-sol, and the like.

The flow path device 10A serves to transfer the buffy coat inside the plurality of BC bags 14 into one pooling bag 18 and collect the buffy coat therein, together with transferring the platelet additive solution inside the medicinal solution bag 16 into the pooling bag 18. A detailed configuration of the flow path device 10A will be described later.

The pooling bag 18 is a bag for accommodating the buffy coat, which has been guided from the plurality of BC bags 14, and the platelet additive solution, which has been guided from the medicinal solution bag 16. One end of a first tube 24 is connected to the pooling bag 18. The other end of the first tube 24 is connected to the filter 20.

The filter 20 removes leukocytes from the liquid (a mixed liquid containing the buffy coat and the platelet additive solution) accommodated in the pooling bag 18. One end of a second tube 26 is connected to the filter 20. The other end of the second tube 26 is connected to the blood platelet bag 22.

The blood platelet bag 22 is a bag for accommodating the blood platelets obtained by removing the leukocytes from the buffy coat. A non-illustrated bag for venting air is connected to the blood platelet bag 22 via a tube 28.

As shown in FIGS. 1 to 3, the flow path device 10A is equipped with a plurality of introduction tubes 30, a plurality of introduction port members 34 (first port members), a rectangular flow path formation member 36 (device main body), and a single lead-out port member 38 (second port member) and a single lead-out tube 40.

In the present embodiment, seven introduction tubes 30 are provided. Ends of six introduction tubes 30 are connected respectively to the six BC bags 14, and an end of a single introduction tube 30 is connected to a sealing member 17 of the medicinal solution bag 16. It should be noted, in the description given below, the introduction tube 30 that is connected to the medicinal solution bag 16 may be referred to as an introduction tube 30a.

The sealing member 17 is formed in a manner so that the interior of the medicinal solution bag 16 and the interior of the introduction tube 30a are placed in communication with each other by the sealing member 17 being subjected to a breaking operation. A clamp 300 for opening and closing an internal hole of the introduction tube 30a is provided in the introduction tube 30a.

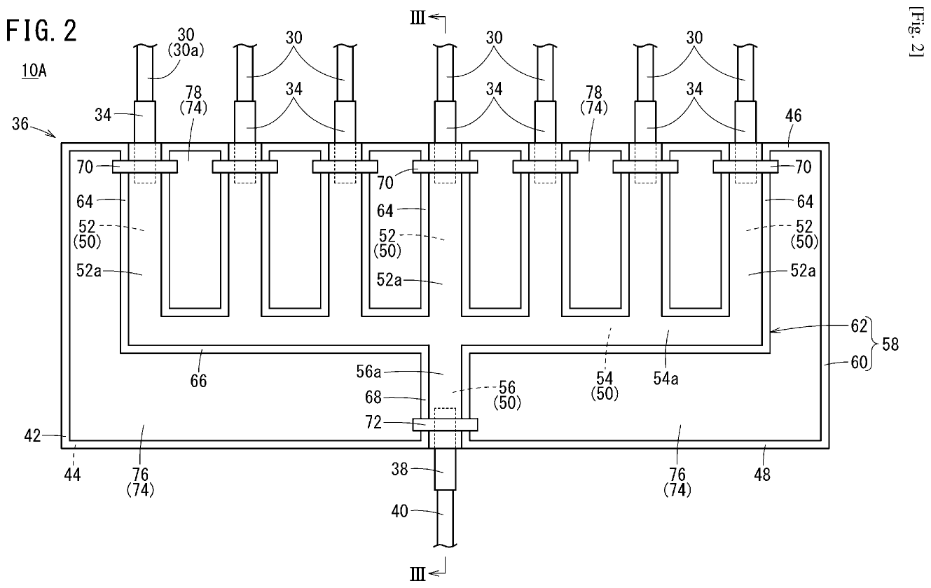

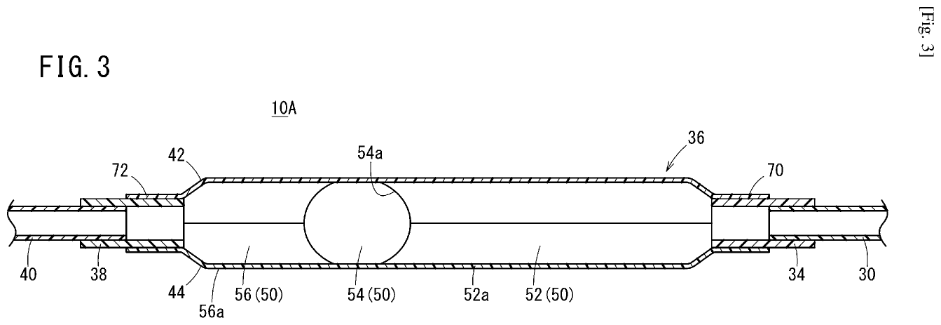

In FIGS. 2 and 3, the flow path formation member 36 is formed by a soft material in a sheet shape. More specifically, the flow path formation member 36 includes a first sheet 42 and a second sheet 44 formed of a soft material. As examples of such a soft material, there may be cited vinyl chloride, polyolefin, polyurethane, and the like. As examples of a vinyl chloride plasticizer, there may be cited diisononylcyclohexane-1,2-dicarboxylate, bis-2-ethylhexyl phthalate, and the like.

The first sheet 42 and the second sheet 44 are joined together mutually in a state of being superimposed in a thickness direction. As examples of the means for joining the first sheet 42 and the second sheet 44, there may be cited fusion bonding (high frequency fusion bonding, thermal fusion bonding, etc.), adhesion, and the like.

As shown in FIG. 2, the plurality of introduction port members 34 are disposed at a first end portion 46, which is one elongate side of the flow path formation member 36. The introduction port members 34 are formed in cylindrical shapes by a hard material (for example, vinyl chloride, polyolefin, polyurethane, or the like). In the present embodiment, seven of the introduction port members 34 are provided. The plurality of introduction port members 34 are arranged at equal intervals in the longitudinal direction of the flow path formation member 36. The centrally positioned introduction port member 34 is located at a central portion in the longitudinal direction of the flow path formation member 36. The other ends of the introduction tubes 30 are connected respectively to the plurality of introduction port members 34.

A single lead-out port member 38 is provided on a second end portion 48, which is another elongate side of the flow path formation member 36. The lead-out port member 38 is configured in the same manner as the introduction port members 34. The lead-out port member 38 is located at a central portion in the longitudinal direction of the flow path formation member 36. One end of the lead-out tube 40 is connected to the lead-out port member 38. Another end of the lead-out tube 40 is connected to the pooling bag 18. A clamp 302 for opening and closing an internal hole of the lead-out tube 40 is provided on the lead-out tube 40 (see FIG. 1).

Liquid flow paths 50 (flow paths) through which at least one of the buffy coat (biological component) and a platelet additive solution (medicinal solution) flows are formed in the flow path formation member 36. The liquid flow paths 50 are formed between the first sheet 42 and the second sheet 44. The liquid flow paths 50 include a plurality of first flow paths 52, a single intermediate flow path 54, and a single second flow path 56.

In the present embodiment, seven of the first flow paths 52 are provided. The first flow paths 52 extend in straight line shapes from the first end portion 46 toward the second end portion 48 of the flow path formation member 36. The introduction port members 34 are provided in the respective first flow paths 52. The intermediate flow path 54 extends along the longitudinal direction of the flow path formation member 36. Extending ends of the plurality of first flow paths 52 are connected to the intermediate flow path 54. The second flow path 56 extends in a straight line shape from the intermediate flow path 54 to the second end portion 48 of the flow path formation member 36. The lead-out port member 38 is provided in the second flow path 56.

More specifically, the plurality of first flow paths 52 are located on a side opposite to the second flow path 56 (the side of the first end portion 46) with respect to the intermediate flow path 54. The plurality of first flow paths 52 are arranged alongside one another in the direction of extension of the intermediate flow path 54 in a state of being separated from each other. The second flow path 56 communicates with a central portion in the direction of extension of the intermediate flow path 54.

The flow path formation member 36 includes a plurality of first flow path wall portions 52a that form the plurality of first flow paths 52, an intermediate flow path wall portion 54a that forms the intermediate flow path 54, and a second flow path wall portion 56a that forms the second flow path 56. The plurality of first flow path wall portions 52a, the intermediate flow path wall portion 54a, and the second flow path wall portion 56a bulge in convex shapes in the thickness direction of the flow path formation member 36 in a natural state (see FIG. 3).

Sealed portions 58, which are fusion-bonded portions between the first sheet 42 and the second sheet 44, are formed in the flow path formation member 36. The sealed portions 58 include an outer edge sealed portion 60 and flow path sealed portions 62. The outer edge sealed portion 60 joins an outer edge portion of the first sheet 42 and an outer edge portion of the second sheet 44 to each other. The flow path sealed portions 62 join the first sheet 42 and the second sheet 44 together in a liquid-tight manner on both sides of the liquid flow paths 50. More specifically, the flow path sealed portions 62 include first flow path sealed portions 64 provided respectively on both sides of each of the plurality of first flow paths 52, intermediate flow path sealed portions 66 provided on both sides of the intermediate flow path 54, and second flow path sealed portions 68 provided on both sides of the second flow path 56.

The first flow path sealed portions 64 extend along the entire length of the first flow paths 52. The first flow path sealed portions 64 are connected to the outer edge sealed portion 60 and the intermediate flow path sealed portions 66. The intermediate flow path sealed portions 66 extend along the entire length of the intermediate flow path 54. The intermediate flow path sealed portions 66 are connected to the first flow path sealed portions 64 and the second flow path sealed portions 68. The second flow path sealed portions 68 extend along the entire length of the second flow path 56. The second flow path sealed portions 68 are connected to the intermediate flow path sealed portions 66 and the outer edge sealed portion 60.

A plurality of first fixing members 70 for fixing the plurality of introduction port members 34, and a second fixing member 72 for fixing the lead-out port member 38 are provided on the flow path formation member 36. The first fixing members 70 extend in a direction perpendicular to (intersecting with) the direction of extension of the introduction port members 34. The first sheet 42 and the second sheet 44 are fixed to each other at both ends of the first fixing members 70. In central parts of the first fixing members 70, the first sheet 42 and the introduction port members 34 are fixed (sealed) to each other, and the second sheet 44 and the introduction port members 34 are fixed (sealed) to each other (see FIG. 3).

The second fixing member 72 extends in a direction perpendicular to (intersecting with) the direction of extension of the lead-out port member 38. The first sheet 42 and the second sheet 44 are fixed to each other at both respective ends of the second fixing member 72. In a central part of the second fixing member 72, the first sheet 42 and the introduction lead-out port member 38 are fixed (sealed) to each other, and the second sheet 44 and the lead-out port member 38 are fixed (sealed) to each other (see FIG. 3).

Non-sealed portions 74 where the first sheet 42 and the second sheet 44 are not joined (fusion bonded) to each other are provided at portions other than the liquid flow paths 50, within the flow path formation member 36. The non-sealed portions 74 do not bulge outwardly in the thickness direction. The non-sealed portions 74 are formed to be thicker than the sealed portions 58. The non-sealed portions 74 are disposed along the first flow path sealed portions 64, the intermediate flow path sealed portions 66, and the second flow path sealed portions 68. Stated otherwise, the non-sealed portions 74 are connected to the first flow path sealed portions 64, the intermediate flow path sealed portions 66, and the second flow path sealed portions 68.

The non-sealed portions 74 include first non-sealed portions 76 and second non-sealed portions 78. The first non-sealed portions 76 are surrounded by the most outwardly positioned first flow path sealed portions 64, the intermediate flow path sealed portions 66, the second flow path sealed portions 68, and the outer edge sealed portion 60. The second non-sealed portions 78 are each positioned between the first flow path sealed portions 64 that lie adjacent to each other. The outer edge sealed portion 60 extends so as to surround the non-sealed portions 74.

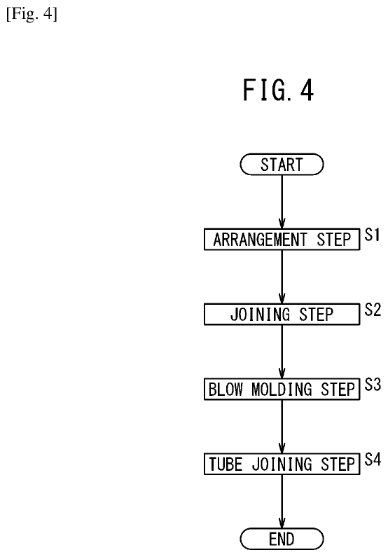

As shown in FIG. 4, the method of manufacturing the flow path device 10A provided with the above-described configuration includes an arrangement step, a joining step, a blow molding step, and a tube joining step.

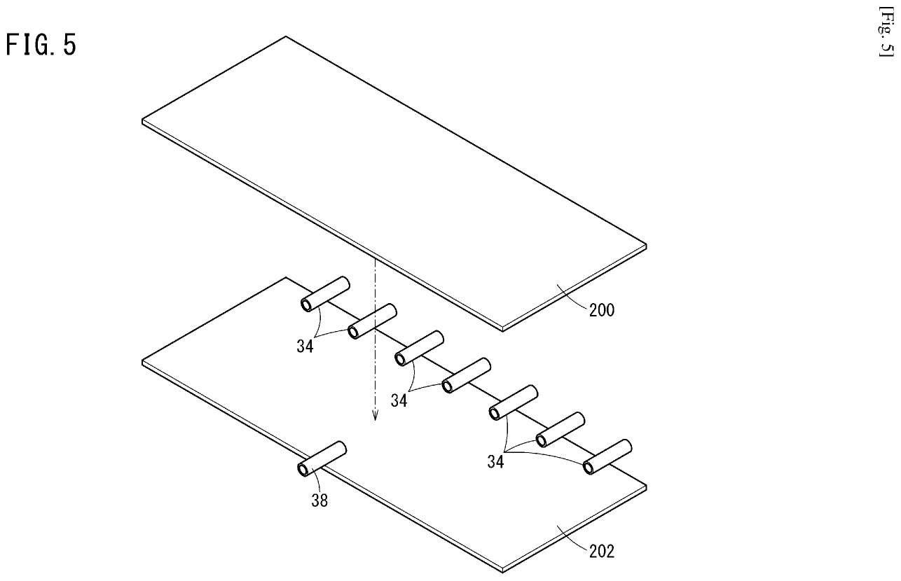

In the arrangement step (step S1 of FIG. 4), as shown in FIG. 5, the plurality of (seven) introduction port members 34 and one lead-out port member 38 are set so as to be arranged between a rectangular first material sheet 200 that is formed by a soft material, and a rectangular second material sheet 202 that is formed by a soft material. The introduction port members 34 are arranged between one elongate side of the first material sheet 200 and one elongate side of the second material sheet 202. The lead-out port member 38 is arranged between another elongate side of the first material sheet 200 and another elongate side of the second material sheet 202.

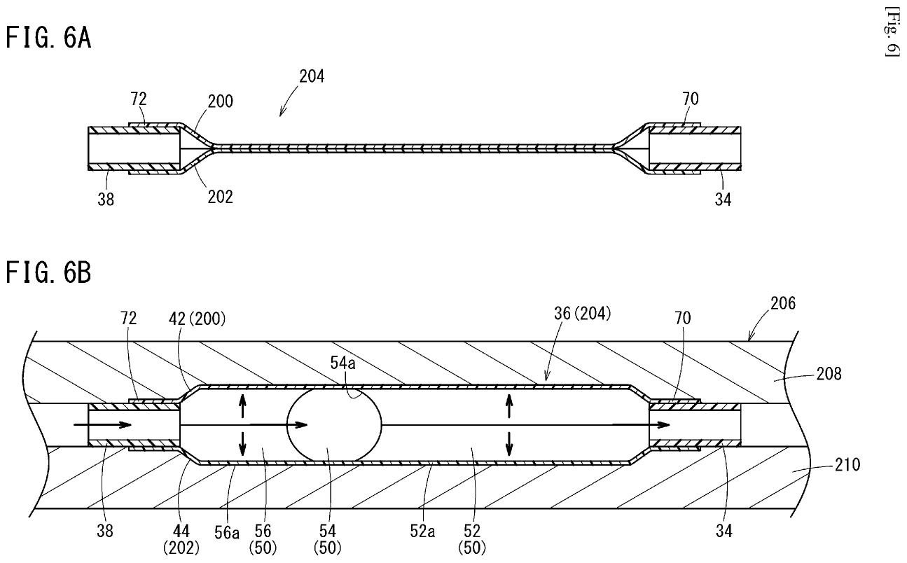

In the joining step (step S2 of FIG. 4), as shown in FIG. 6A, the plurality of introduction port members 34 are joined to the first material sheet 200 and the second material sheet 202, and the lead-out port member 38 is joined to the first material sheet 200 and the second material sheet 202. In this case, during such joining, a high frequency fusion bonding device, or a heat fusion bonding device or the like is used. The first fixing members 70 and the second fixing member 72 are formed on an intermediate product 204 that is obtained by the joining step.

In addition, in the blow molding step (step S3 of FIG. 4), the intermediate product 204 is placed at a predetermined position in a pair of molds 208 and 210 that constitute the sheet joining device 206, as shown in FIG. 6B. In the present embodiment, the sheet joining device 206 is a high frequency fusion bonding device. The sheet joining device 206 may also be a heat fusion bonding device or the like. On molding surfaces of the pair of molds 208 and 210, there are provided grooves for forming portions surrounding the liquid flow paths 50 of the flow path formation member 36 (the first flow path wall portions 52a, the intermediate flow path wall portion 54a, and the second flow path wall portion 56a).

In the blow molding step, the first material sheet 200 and the second material sheet 202 are sandwiched between the molds 208 and 210, and the first material sheet 200 and the second material sheet 202 are joined to each other, together with blow molding being performed so as to form the liquid flow paths 50.

More specifically, the pair of molds 208 and 210 are closed, the first material sheet 200 and the second material sheet 202 are overlapped, and predetermined locations of the first material sheet 200 and the second material sheet 202 are subjected to high frequency fusion bonding so as to form the liquid flow paths 50 (the first flow paths 52, the intermediate flow path 54, and the second flow path 56). At this time, air is blown out from a non-illustrated blow nozzle, and in the first material sheet 200 and the second material sheet 202, locations thereof corresponding to the grooves provided in the molds 208 and 210 are inflated, whereby the liquid flow paths 50 (the first flow paths 52, the intermediate flow path 54, and the second flow path 56) are formed.

After completion of the blow molding step, the blow nozzle is pulled out from the flow path formation member 36. Next, the pair of molds 208 and 210 are opened, and the flow path formation member 36 is taken out in the form of a molded product.

Thereafter, in the tube joining step (step S4 of FIG. 4), the introduction tubes 30 are joined respectively to the plurality of introduction port members 34, and the lead-out tube 40 is joined to the lead-out port member 38. Consequently, by carrying out the aforementioned steps, the flow path device 10A is manufactured.

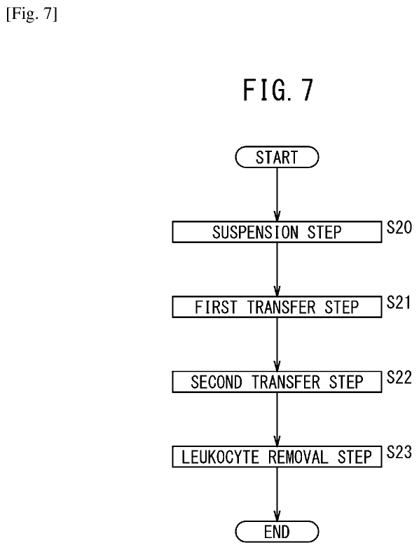

Next, a blood platelet collection method for obtaining blood platelets in which leukocytes have been removed from the buffy coat, using the biological component bag system 12 will be described. As shown in FIG. 7, the blood platelet collection method includes a suspension step, a first transfer step, a second transfer step, and a leukocyte removal step.

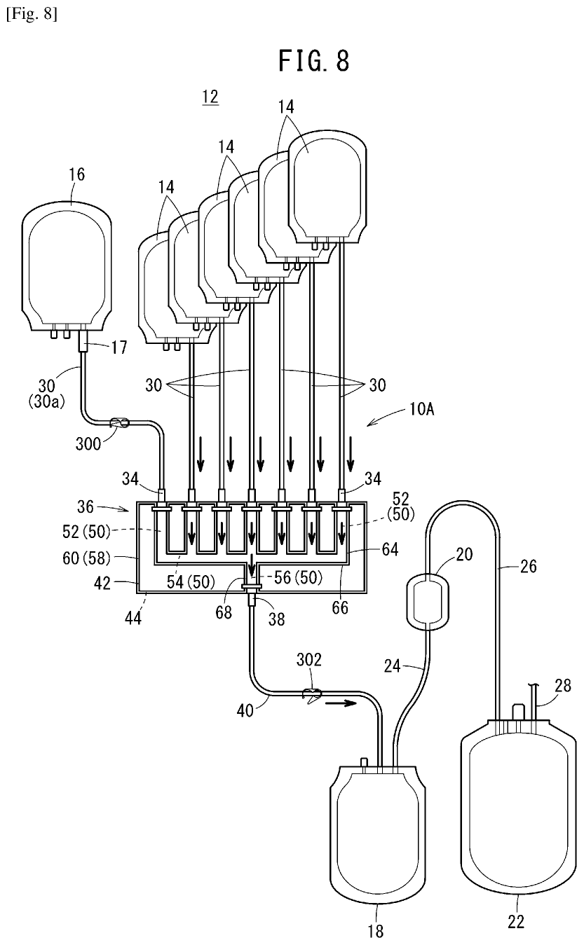

As shown in FIG. 8, at first, in the suspension step (step S20 of FIG. 7), the plurality of BC bags 14 and the medicinal solution bag 16 are suspended on a non-illustrated suspension platform. At this time, the flow path device 10A is suspended by the introduction tubes 30. In greater detail, the blood platelet bag 22 is located at a more downward (vertically downward) position than the plurality of BC bags 14 and the medicinal solution bag 16.

Next, in the first transfer step (step S21 of FIG. 7), the clamp 302 is opened. Upon doing so, due to the action of gravity (difference in elevation), the buffy coat inside the plurality of BC bags 14 is transferred into the interior of the pooling bag 18 via the plurality of introduction tubes 30, the plurality of introduction port members 34, the plurality of first flow paths 52, the intermediate flow path 54, the second flow path 56, the lead-out port member 38, and the lead-out tube 40.

Upon completion of the first transfer step, then in the second transfer step (step S22 of FIG. 7), the sealing member 17 of the medicinal solution bag 16 is broken together with opening the clamp 300. Upon doing so, due to the action of gravity (difference in elevation), the platelet additive solution inside the medicinal solution bag 16 is transferred into the interior of the pooling bag 18 via the introduction tube 30a, the introduction port member 34, the intermediate flow path 54, the second flow path 56, the lead-out port member 38, and the lead-out tube 40. Consequently, the blood plasma contained in the buffy coat inside the pooling bag 18 is replaced by the platelet additive solution. Thereafter, the lead-out tube 40 is fusion bonded and sealed by a sealer or the like, and the lead-out tube 40 is cut at the sealed portion.

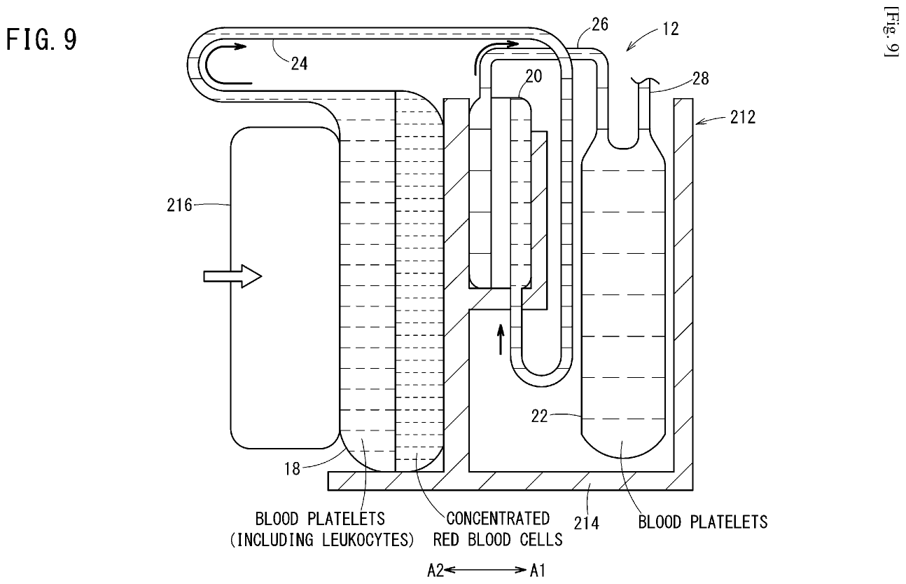

Thereafter, in the leukocyte removal step (step S23 of FIG. 7), as shown in FIG. 9, the pooling bag 18 of the biological component bag system 12 is mounted in a non-illustrated cassette, and the cassette is mounted in an insert unit 214 of a centrifugal separation and transfer device 212. At this time, the filter 20 is positioned more on an outer side in a radial direction (in the direction of the arrow A1) of the centrifugal separation and transfer device 212 than the pooling bag 18, and the blood platelet bag 22 is positioned more on an outer side in the radial direction (in the direction of the arrow A1) of the centrifugal separation and transfer device 212 than the filter 20.

Thereafter, a non-illustrated centrifugation drum of the centrifugal separation and transfer device 212 is rotated. Upon doing so, by the buffy coat accommodated in the pooling bag 18 receiving the centrifugal force, the concentrated red blood cells move to the outer side in the radial direction of the centrifugal separation and transfer device 212, whereas the blood platelets (including the leukocytes) move inwardly in the radial direction (in the direction of the arrow A2) of the centrifugal separation and transfer device 212, and the buffy coat is separated into two layers. Subsequently, while the centrifugal force continues being applied to the pooling bag 18, a pressing member 216 is displaced in a direction of the centrifugal force, and in particular, in a radial outward direction (in the direction of the arrow A1) of the centrifugal separation and transfer device 212 to thereby press the pooling bag 18.

Upon doing so, the blood platelets (including the leukocytes) in the interior of the pooling bag 18 flow through the filter 20 via the first tube 24. At this time, the leukocytes contained in the blood platelets are removed (trapped) by the filter 20. The blood platelets from which the leukocytes have been removed are accommodated and stored in the blood platelet bag 22 via the second tube 26. As a result, blood platelets that do not contain any leukocytes can be obtained.

In this case, the flow path device 10A and the biological component bag system 12 according to the present embodiment exhibit the following advantageous effects.

In the flow path device 10A, the liquid flow paths 50 include the plurality of first flow paths 52, the intermediate flow path 54 that communicates with the plurality of first flow paths 52, and the second flow path 56 that communicates with the intermediate flow path 54. The flow path formation member 36 includes the first sheet 42 that is formed by a soft material, and the second sheet 44 that is superimposed on the first sheet 42 and is formed by a soft material. The liquid flow paths 50 are formed between the first sheet 42 and the second sheet 44, and on both sides of each of the liquid flow paths 50 within the flow path formation member 36, the flow path sealed portions 62 are provided which join the first sheet 42 and the second sheet 44 to each other in a liquid-tight manner.

In accordance with such a configuration, the liquid flow paths 50, which include the plurality of first flow paths 52, the intermediate flow path 54, and the second flow path 56, are formed between the first sheet 42 and the second sheet 44. Therefore, compared to a conventional product in which a plurality of branch connectors and a plurality of tubes are used, the number of component parts can be reduced. Further, the flow path sealed portions 62 that join the first sheet 42 and the second sheet 44 to each other in a liquid-tight manner are disposed on both sides of each of the liquid flow paths 50. Consequently, since there is no need to join a large number of component parts, the number of joining steps can be reduced. Thus, a reduction in costs can be achieved.

The plurality of first flow paths 52 are located on a side opposite to the second flow path 56 with respect to the intermediate flow path 54.

In accordance with such a configuration, the configuration of the flow path device 10A can be simplified.

The plurality of first flow paths 52 are arranged alongside one another in the direction of extension of the intermediate flow path 54 in a state of being separated from each other.

In accordance with such a configuration, the configuration of the flow path device 10A can be further simplified.

The second flow path 56 communicates with a central portion of the intermediate flow path 54 in the direction of extension thereof.

In accordance with such a configuration, the buffy coat (biological component) that is guided to each of the first flow paths 52 can be made to flow efficiently through the second flow path 56.

The plurality of first flow paths 52 is greater than or equal to three.

In accordance with such a configuration, the cost of the flow path device 10A can be further reduced in comparison with a conventional product.

The flow path sealed portions 62 include the first flow path sealed portions 64 provided respectively on both sides of each of the plurality of first flow paths 52, the intermediate flow path sealed portions 66 provided on both sides of the intermediate flow path 54, and the second flow path sealed portions 68 provided on both sides of the second flow path 56.

In accordance with such a configuration, leakage of liquid from the first flow paths 52, the intermediate flow path 54, and the second flow path 56 can be effectively suppressed.

The non-sealed portions 74 where the first sheet 42 and the second sheet 44 are not joined to each other are provided at positions other than the flow paths, within the flow path formation member, and the non-sealed portions 74 are provided along the first flow path sealed portions 64, the intermediate flow path sealed portions 66, and the second flow path sealed portions 68.

In accordance with such a configuration, the first flow path sealed portions 64, the intermediate flow path sealed portions 66, and the second flow path sealed portions 68 can be protected by the material of the non-sealed portions 74.

The outer edge sealed portion 60, which extends in a manner so as to surround the non-sealed portions 74 and which joins the first sheet 42 and the second sheet 44 to each other, is provided in the flow path formation member 36.

In accordance with such a configuration, the non-sealed portions 74 can be protected by the outer edge sealed portion 60.

(First Modification)

Next, a flow path device 10B according to a first modification will be described. In the flow path device 10B according to the present modification, constituent elements thereof, which are the same as those of the above-described flow path device 10A, are designated with the same reference characters, and detailed description of such features is omitted. The same convention applies to a flow path device 10C according to a second modification and a flow path device 10D according to a third modification, which will be described later.

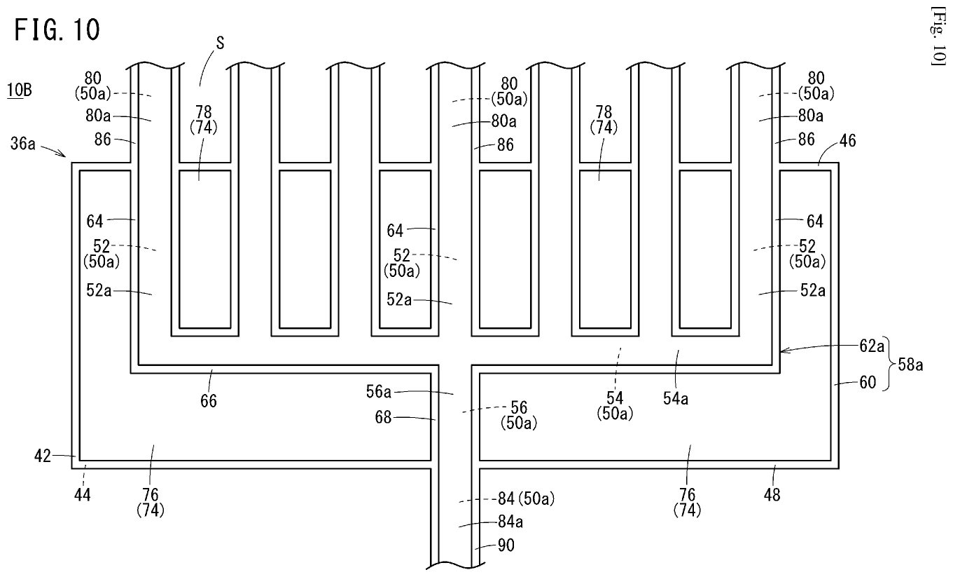

As shown in FIG. 10, the liquid flow paths 50a (flow paths) of the flow path formation member 36a of the flow path device 10B include a plurality of (seven) introduction flow paths 80 (communication paths) in communication respectively with a plurality of (seven) first flow paths 52, and a lead-out flow path 84 (connecting flow path) in communication with the second flow path 56. The flow path formation member 36a includes a plurality of introduction flow path wall portions 80a (communication path wall portions) that form the plurality of introduction flow paths 80, and a plurality of lead-out flow path wall portions 84a (connecting flow path wall portions) that form the lead-out flow path 84.

The sealed portions 58a include the outer edge sealed portion 60 and the flow path sealed portions 62a. The flow path sealed portions 62a are equipped with introduction flow path sealed portions 86 (communication path sealed portions) provided respectively on both sides of each of the plurality of introduction flow paths 80, and lead-out flow path sealed portions 90 (connecting flow path sealed portions) provided on both sides of the lead-out flow path 84.

The introduction flow path sealed portions 86 and the lead-out flow path sealed portions 90 extend along the outer shape of the flow path formation member 36a. More specifically, the non-sealed portions 74 do not exist on outer sides of the introduction flow path sealed portions 86 and on outer sides of the lead-out flow path sealed portions 90. Therefore, spaces S (spaces) that are extended along the introduction flow path sealed portions 86 are formed between the introduction flow path sealed portions 86 that lie adjacent to each other.

In the flow path device 10B, the introduction port members 34 and the lead-out port member 38 described above are omitted. Further, six introduction flow path wall portions 80a are provided instead of the aforementioned six introduction tubes 30, and therefore are connected respectively to the plurality of BC bags 14. One of the introduction flow path wall portions 80a is provided instead of the aforementioned single introduction tube 30a, and therefore is connected to the medicinal solution bag 16. The lead-out flow path wall portion 84a is provided instead of the aforementioned lead-out tube 40, and therefore is connected to the pooling bag 18. A clamp 300 (see FIG. 1) is provided on the introduction flow path wall portion 80a connected to the medicinal solution bag 16, and a clamp 302 (see FIG. 1) is provided on the lead-out flow path wall portion 84a.

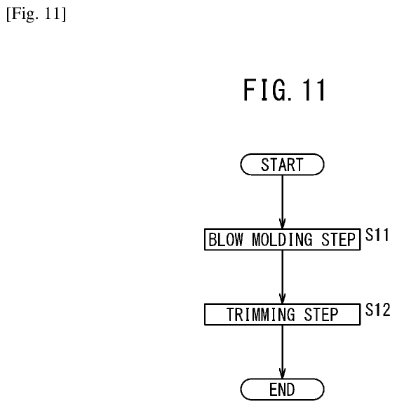

In the method of manufacturing such a flow path device 10B, as shown in FIG. 11, a blow molding step and a trimming step are carried out.

In the blow molding step (step S11 of FIG. 11), the same process as in the blow molding step (step S3 of FIG. 4) described above is performed. More specifically, the first material sheet 200 and the second material sheet 202 are superimposed on each other, and the first material sheet 200 and the second material sheet 202 are subjected to high frequency fusion bonding and blow molding at predetermined positions so as to form the liquid flow paths 50a (the introduction flow paths 80, the first flow paths 52, the intermediate flow path 54, the second flow path 56, and the lead-out flow path 84).

In the trimming step (step S12 in FIG. 11), after completion of the blow molding step, portions of the first material sheet 200 and the second material sheet 202 are trimmed (cut). More specifically, the first material sheet 200 and the second material sheet 202 are cut along each of the introduction flow path sealed portions 86 and the lead-out flow path sealed portions 90. In addition, within the first material sheet 200 and the second material sheet 202, portions thereof between the adjacent introduction flow path sealed portions 86, and portions on the outer side of the lead-out flow path sealed portions 90 are removed. Consequently, by carrying out the aforementioned steps, the flow path device 10B is manufactured.

In the present modification, the liquid flow paths 50a include the plurality of introduction flow paths 80 that communicate respectively with the plurality of first flow paths 52. The flow path sealed portions 62a include the introduction flow path sealed portions 86 provided respectively on both sides of each of the plurality of introduction flow paths 80. The spaces S that are extended along the introduction flow path sealed portions 86 are formed between the introduction flow path sealed portions 86 that lie adjacent to each other.

In accordance with such a configuration, since the plurality of introduction flow paths 80 can be integrally provided in the flow path formation member 36a, the cost of the flow path device 10B can be further reduced. Further, since the spaces S are formed between the introduction flow path sealed portions 86 that lie adjacent to each other, the plurality of introduction flow paths 80 can be freely arranged without interfering with each other. Furthermore, the clamp 300 can be easily attached to the introduction flow path 80.

The liquid flow paths 50a include the lead-out flow path 84 that communicates with the second flow path 56. The flow path sealed portions 62a include the lead-out flow path sealed portions 90 provided on both sides of the lead-out flow path 84. The lead-out flow path sealed portions 90 extend along the outer shape of the flow path formation member 36a.

In accordance with such a configuration, since the lead-out flow path 84 can be integrally provided in the flow path formation member 36a, the cost of the flow path device 10B can be further reduced. Further, since the lead-out flow path sealed portions 90 extend along the outer shape of the flow path formation member 36a, positional adjustment of the lead-out flow path 84 can be easily performed. Furthermore, the clamp 302 can be easily attached to the lead-out flow path 84.

In the present modification, in the flow path device 10B, the plurality of introduction flow paths 80 may be replaced by the above-described introduction tubes 30. Further, in the flow path device 10B, the lead-out flow path 84 may be replaced by the above-described lead-out tube 40.

(Second Modification)

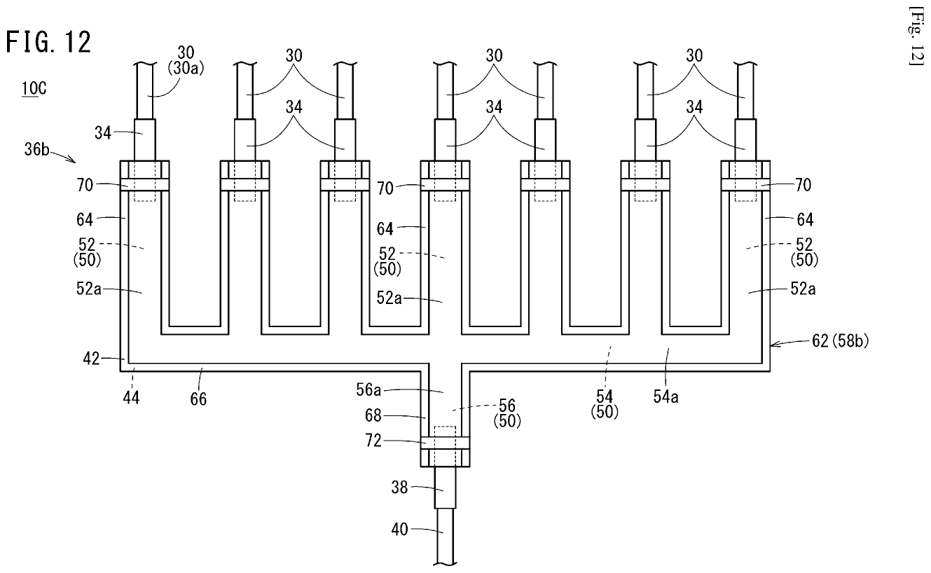

Next, a flow path device 10C according to a second modification will be described. As shown in FIG. 12, in the flow path formation member 36b of the flow path device 10C according to the present modification, trimming (cutting) is carried out on the aforementioned non-sealed portions 74. Stated otherwise, the sealed portions 58b of the flow path formation member 36b include the flow path sealed portions 62 without including the above-described outer edge sealed portion 60. The first flow path sealed portions 64, the intermediate flow path sealed portions 66, and the second flow path sealed portions 68 extend along the outer shape of the flow path formation member 36b.

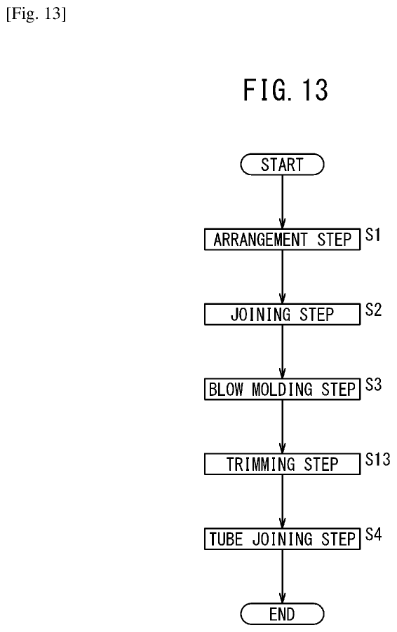

As shown in FIG. 13, the method of manufacturing such a flow path device 10C includes an arrangement step, a joining step, a blow molding step, a trimming step, and a tube joining step. Moreover, since descriptions of the arrangement step (step S1), the joining step (step S2), the blow molding step (step S3), and the tube joining step (step S4) have already been described previously, they are omitted.

In the trimming step (step S13 of FIG. 13), the first material sheet 200 and the second material sheet 202 are cut along each of the first flow path sealed portions 64, the intermediate flow path sealed portions 66, and the second flow path sealed portions 68. In addition, within the first material sheet 200 and the second material sheet 202, portions thereof between the adjacent first flow path sealed portions 64, and portions on outer sides of the first flow path sealed portions 64 positioned on the outermost side, the intermediate flow path sealed portions 66, and the second flow path sealed portions 68 are removed. Consequently, by carrying out the aforementioned steps, the flow path device 10C is manufactured.

In the present modification, the first flow path sealed portions 64, the intermediate flow path sealed portions 66, and the second flow path sealed portions 68 extend along the outer shape of the flow path formation member 36b.

In accordance with such a configuration, the flow path formation member 36b can be made smaller in size.

In the present modification, as in the flow path device 10B according to the first modification, the introduction flow paths 80 may be provided instead of the plurality of introduction tubes 30. Further, in the present modification, as in the flow path device 10B according to the first modification, the lead-out flow path 84 may be provided instead of the lead-out tube 40.

(Third Modification)

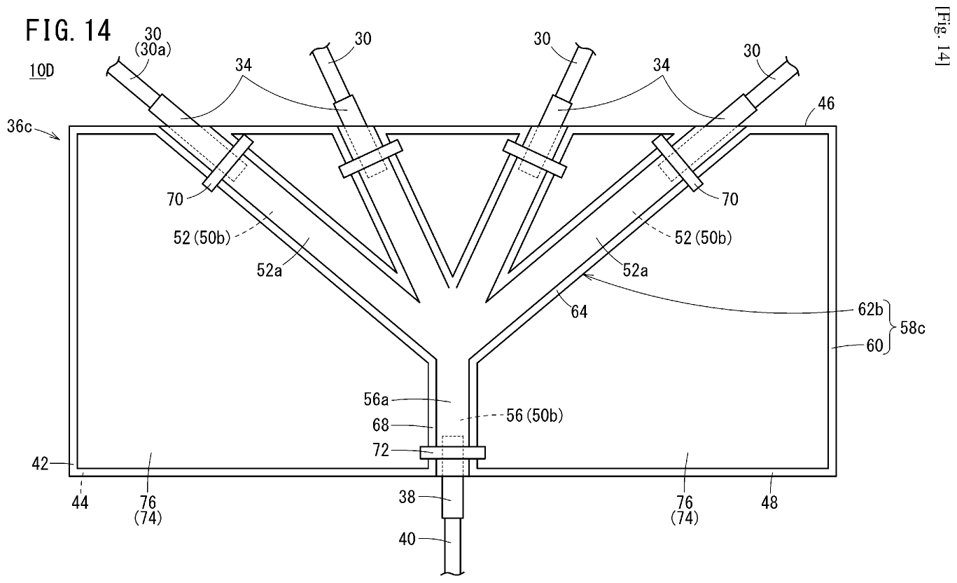

Next, a flow path device 10D according to a third modification will be described. As shown in FIG. 14, the liquid flow paths 50b (flow paths) of the flow path formation member 36c of the flow path device 10D according to the present modification include a plurality of (four) first flow paths 52, and a second flow path 56 that communicates directly with the plurality of first flow paths 52. Stated otherwise, the liquid flow paths 50b do not include the above-described intermediate flow path 54. The plurality of first flow path wall portions 52a are directly connected to the second flow path wall portion 56a.

The sealed portions 58c include the outer edge sealed portion 60 and the flow path sealed portions 62b. The flow path sealed portions 62b include the first flow path sealed portions 64, and the second flow path sealed portions 68 that are directly connected to the first flow path sealed portions 64.

In the flow path device 10D according to the present modification, the same effects are exhibited as those of the above-described flow path device 10A.

In the present modification, as in the flow path device 10B according to the first modification, the introduction flow paths 80 may be provided instead of the plurality of introduction tubes 30. Further, in the present modification, as in the flow path device 10B according to the first modification, the lead-out flow path 84 may be provided instead of the lead-out tube 40.

The present invention is not limited to the above-described embodiment, and various modifications may be adopted within a range that does not depart from the essence and gist of the present invention.

The configurations of the flow paths formed in the flow path formation members 36 and 36a to 36c, and the number and arrangement of the bags (the BC bags 14, the medicinal solution bag 16, and the pooling bag 18) that are provided therein are not limited to the configurations described and illustrated above, and modifications may be made thereto in accordance with the type of biological component and the method of use. Further, the number of the introduction tubes 30, the number of the introduction flow paths 80, and the number of the first flow paths 52 are not limited to seven or four, and can be appropriately changed insofar as a plurality of such members are provided.

In the biological component bag system 12, sealing members, which block communication between the interiors of the BC bags 14 and the interiors of the introduction tubes 30 in an initial state, may be provided in each of the BC bags 14. The sealing members are formed in a manner so that the interiors of the BC bags 14 and the interiors of the introduction tubes 30 are placed in communication with each other by being subjected to a breaking operation. The introduction tubes 30 may be directly fusion bonded to the flow path formation member 36 or 36c without using the introduction port members 34. Further, the lead-out tube 40 may be directly fusion bonded to the flow path formation member 36 or 36c without using the lead-out port member 38.

The flow path devices 10A to 10D can also be used so as to cause the biological component from the second flow path 56 to branch off into the plurality of first flow paths 52 via the intermediate flow path 54.

The above-described embodiment can be summarized in the following manner.

The above-described embodiment is characterized by the flow path device (10A to 10D) including the flow path formation member (36, 36a to 36c) in which there are formed the flow paths (50, 50a, 50b) that allow at least one of a biological component and a medicinal solution to flow therethrough, the flow path formation member including the first sheet (42) formed by a soft material, and the second sheet (44) formed by a soft material and that is superimposed on the first sheet, wherein the flow paths are formed between the first sheet and the second sheet, the flow paths including the plurality of first flow paths (52), and the second flow path (56) that communicates with the plurality of first flow paths, wherein the flow path sealed portions (62, 62a, 62b) that join the first sheet and the second sheet to each other in a liquid-tight manner are provided on both sides of each of the flow paths within the flow path formation member.

In the above-described flow path device, the flow paths may include the intermediate flow path (54) that places the plurality of first flow paths and the second flow path in communication with each other.

In the above-described flow path device, the plurality of first flow paths may be located on an opposite side of the second flow path with respect to the intermediate flow path.

In the above-described flow path device, the plurality of first flow paths may be arranged alongside one another in the direction of extension of the intermediate flow path in a state of being separated from each other.

In the above-described flow path device, the second flow path may communicate with a central portion of the intermediate flow path in the direction of extension thereof.

In the above-described flow path device, the plurality of first flow paths is greater than or equal to three.

In the above-described flow path device, the flow path sealed portions may include the first flow path sealed portions (64) provided on both sides of each of the plurality of first flow paths, the intermediate flow path sealed portions (66) provided on both sides of the intermediate flow path, and the second flow path sealed portions (68) provided on both sides of the second flow path.

In the above-described flow path device, the non-sealed portions where the first sheet and the second sheet are not joined to each other may be provided at positions other than the flow paths, within the flow path formation member, and the non-sealed portions (74) may be provided along the first flow path sealed portions, the intermediate flow path sealed portions, and the second flow path sealed portions.

In the above-described flow path device, the outer edge sealed portion (60), which extends in a manner so as to surround the non-sealed portions and joins the first sheet and the second sheet to each other, may be provided in the flow path formation member.

In the above-described flow path device, the flow paths may include the plurality of communication paths (80 and 82) that communicate respectively with the plurality of first flow paths, and the flow path sealed portions may include the communication path sealed portions (86 and 88) provided on both sides of each of the plurality of communication paths, and the spaces (S) that extend along the communication path sealed portions may be formed between the communication path sealed portions that lie adjacent to each other.

In the above-described flow path device, the flow paths may include the connecting flow path (84) in communication with the second flow path, the flow path sealed portions may include the connecting flow path sealed portions (90) provided on both sides of the connecting flow path, and the connecting flow path sealed portions may extend along the outer shape of the flow path formation member.

In the above-described flow path device, the first flow path sealed portions, the intermediate flow path sealed portions, and the second flow path sealed portions may extend along the outer shape of the flow path formation member.

In the flow path sealed portions, there may be provided the plurality of first port members (34) that form ports in communication respectively with the plurality of first flow paths, and a single second port member (38) that forms a port in communication with the second flow path.

The above-described embodiment is characterized by the biological component bag system (12) configured to collect a desired biological component from a biological fluid, the biological component bag system including the plurality of first bags (14) in which the biological fluid is accommodated, the flow path device to which the plurality of first bags are connected, and the second bag (18) that accommodates the biological component guided from the plurality of first bags via the flow path device, wherein the flow path device includes the flow path formation member in which there are formed the flow paths that allow at least one of the biological fluid and a medicinal solution to flow therethrough, the flow path formation member including the first sheet formed by a soft material, and the second sheet formed by a soft material and that is superimposed on the first sheet, wherein the flow paths are formed between the first sheet and the second sheet, the flow paths including the plurality of first flow paths, and the single second flow path that communicates with the plurality of first flow paths, wherein the flow path sealed portions that join the first sheet and the second sheet to each other in a liquid-tight manner are provided on both sides of each of the flow paths within the flow path formation member.

In the above-described biological component bag system, there may further be provided the medicinal solution bag (16) connected to the flow path device and in which the medicinal solution is accommodated, the filter (20) for removing the predetermined biological component from the liquid containing the medicinal solution and the biological component accommodated inside the second bag via the flow path device, and the third bag (22) for accommodating the biological component that has passed through the filter.

In the above-described biological component bag system, the buffy coat as the biological fluid may be accommodated in each of the plurality of first bags, the platelet additive solution as the medicinal solution may be accommodated in the medicinal solution bag, the filter may remove leukocytes from the buffy coat, and the blood platelets that have passed through the filter and the platelet additive solution may be accommodated in the third bag.

What is claimed is:

1. A flow path device comprising:
a flow path formation member in which there are formed flow paths configured to allow at least one of a biological fluid and a medicinal solution to flow therethrough;
the flow path formation member comprising:
a first sheet formed by a soft material; and
a second sheet formed by a soft material and that is superimposed on the first sheet;
wherein the flow paths are formed between the first sheet and the second sheet;
the flow paths comprising:
a plurality of first flow paths; and
a single second flow path configured to communicate with the plurality of first flow paths;
wherein flow path sealed portions configured to join the first sheet and the second sheet to each other in a liquid-tight manner are provided on both sides of each of the flow paths within the flow path formation member.

2. The flow path device according to claim 1, wherein the flow paths include an intermediate flow path configured to place the plurality of first flow paths and the second flow path in communication with each other.

3. The flow path device according to claim 2, wherein the plurality of first flow paths are located on an opposite side of the second flow path with respect to the intermediate flow path.

4. The flow path device according to claim 3, wherein the plurality of first flow paths are arranged alongside one another in a direction of extension of the intermediate flow path in a state of being separated from each other.

5. The flow path device according to claim 4, wherein the second flow path communicates with a central portion of the intermediate flow path in the direction of extension thereof.

6. The flow path device according to claim 4, wherein the plurality of first flow paths is greater than or equal to three.

7. The flow path device according to claim 2, wherein the flow path sealed portions comprise:
first flow path sealed portions provided on both sides of each of the plurality of first flow paths;
intermediate flow path sealed portions provided on both sides of the intermediate flow path; and
second flow path sealed portions provided on both sides of the second flow path.

8. The flow path device according to claim 7, wherein:
non-sealed portions where the first sheet and the second sheet are not joined to each other are provided at positions other than the flow paths, within the flow path formation member; and
the non-sealed portions are provided along the first flow path sealed portions, the intermediate flow path sealed portions, and the second flow path sealed portions.

9. The flow path device according to claim 8, wherein an outer edge sealed portion, which is configured to extend in a manner so as to surround the non-sealed portions and join the first sheet and the second sheet to each other, is provided in the flow path formation member.

10. The flow path device according to claim 8, wherein:
the flow paths include a plurality of communication paths configured to communicate with the plurality of first flow paths respectively;
the flow path sealed portions include communication path sealed portions provided on both sides of each of the plurality of communication paths; and
spaces configured to extend along the communication path sealed portions are formed between the communication path sealed portions that lie adjacent to each other.

11. The flow path device according to claim 8, wherein:
the flow paths include a connecting flow path in communication with the second flow path;
the flow path sealed portions include connecting flow path sealed portions provided on both sides of the connecting flow path; and
the connecting flow path sealed portions extend along an outer shape of the flow path formation member.

12. The flow path device according to claim 7, wherein the first flow path sealed portions, the intermediate flow path sealed portions, and the second flow path sealed portions extend along an outer shape of the flow path formation member.

13. The flow path device according to claim 1, wherein the flow path sealed portions comprise:
a plurality of first port members configured to form ports in communication respectively with the plurality of first flow paths; and
a single second port member configured to form a port in communication with the second flow path.

14. A biological component bag system configured to collect a desired biological component from a biological fluid, the biological component bag system comprising:
a plurality of first bags in which the biological fluid is accommodated;
a flow path device to which the plurality of first bags are connected; and
a second bag configured to accommodate the biological component guided from the plurality of first bags via the flow path device;
wherein the flow path device comprises a flow path formation member in which there are formed flow paths configured to allow at least one of the biological fluid and a medicinal solution to flow therethrough;
the flow path formation member comprising:
a first sheet formed by a soft material; and
a second sheet formed by a soft material and that is superimposed on the first sheet;
wherein the flow paths are formed between the first sheet and the second sheet;
the flow paths comprising:
a plurality of first flow paths; and
a single second flow path configured to communicate with the plurality of first flow paths;
wherein flow path sealed portions configured to join the first sheet and the second sheet to each other in a liquid-tight manner are provided on both sides of each of the flow paths within the flow path formation member.

15. The biological component bag system according to claim 14, further comprising:
a medicinal solution bag connected to the flow path device and in which a medicinal solution is accommodated;
a filter configured to remove a predetermined biological component from a liquid containing the medicinal solution and the biological component accommodated inside the second bag via the flow path device; and
a third bag configured to accommodate the biological component that has passed through the filter.

16. The biological component bag system according to claim 15, wherein:
a buffy coat as the biological fluid is accommodated in each of the plurality of first bags;
a platelet additive solution as the medicinal solution is accommodated in the medicinal solution bag;
the filter removes leukocytes from the buffy coat; and
blood platelets that have passed through the filter and the platelet additive solution are accommodated in the third bag.
